(19) United States
(12) Patent Application Publication
(10) Pub. No.: US 2019/0336349 A1
(43) Pub. Date: Nov. 7, 2019

(54) Health Care Device

(71) Applicant: Treena Lynn Scott, Miami, FL (US)

(72) Inventor: Treena Lynn Scott, Miami, FL (US)

(21) Appl. No.: 15/971,926

(22) Filed: May 4, 2018

(51) Int. Cl. A61F 13/02; A61F 13/00

(52) U.S. Cl. CPC A61F 13/0243; A61G 7/05784; A61F 13/00051

(57) ABSTRACT

The invention is a wound care device having substantially rigid frame with a broad base strong enough to support the weight of sheets, blankets and other items used to cover the patient. The base has at least two points of contact with the bed, table or chair that the patient is lying on or sitting in. These points of contact are connected to each other by the bridge like frame and are dispersed widely enough so that the barrier is able to straddle the relevant body parts. At the same time, it is small enough so that the body is able to easily heat a pocket of air created within the barrier and bedclothes. The device is tapered to accommodate the shape of limbs, with the ratio of diameters providing order point data.

### BACKGROUND OF THE INVENTION

### 1. Field of the Invention

The present invention relates to the field of health care. More specifically, it relates to a medical care device focusing on the healing of traumatic type physical injuries which are generally described in the medical literature as wounds, lesions, contusions, and abrasions. We have found in unscientific searching that the use of a variety of terms leads to a more comprehensive description of the prior art.

A person who is hospitalized and has an open injury such as a wound, lesion, contusion abrasion needs to stay warm in order to prevent illness or the worsening of illness, and at the same time it is helpful to expose the open injury to air to accelerate scab formation, which, of course, leads to the healing of the injury. Bedclothes or bandages can slow down the healing process, both by preventing scab formation and by adhering to the wound as it is healing. In addition, after having adhered to an injury, bedclothes or bandages can reopen the injury upon their removal.

The wound care device of the present invention is useful for preventing contact between bedclothes and an open wound without the use of bandages and unlike bandages provides the wound with ventilation while allowing the bedclothes to perform their function of minimizing the loss of body heat. In unscientific search, Applicant located a number of prior art references all of which are more complex than the present invention. These references include Robins et al, U.S. Pat. No. 4,175,297 for an inflatable pillow support, Schild et al, U.S. Pat. No. 4,391,009 for a ventilated body support, Senoue et al, U.S. Pat. No. 4,653,130 for a bed sore preventing apparatus, Kimura et al, U.S. Pat. No. 4,825,486 for a bed sore-preventing air mattress controller, Wapeidus, U.S. Pat. No. 3,653,083 for a bed pad, and Lin, U.S. Pat. No. 6,266,833 for an air bed structure capable of alternate aerating and lying thereon one's side. As noted above, all of these references are more complex which speaks to the prospects for patentability of the present invention by reason of its simplicity.

### SUMMARY OF THE INVENTION

Bearing in mind the foregoing, it is a principle object of the present invention to provide a very simple wound care device to use in conjunction with the healing of traumatic injuries on the external surface of a patient's body.

A related principle object of the present invention is to achieve the foregoing for such injuries as wounds, lesions, contusions, and abrasions.

Another important object of the invention is to maintain separation between bedclothes and/or bandages from the injury to allow aeration thereof.

A related significant object of the invention is to achieve the aeration objective while allowing the bedclothes to perform their function of minimizing loss of body heat.

A further object of the invention is to prevent contact by bedclothes and/or bandages to avoid adhering to the wound, removal of which would reopen the wound.

An additional object of the invention is to permit aeration of the injury to facilitate formation of scabs thereby promoting healing of the wound.

Another object of the invention is to accommodate the shape of limbs of the body by tapering the diameter and/or cross sectional area of the wound care device in accordance with a ratio that can provide device ordering data, i.e., A/B equal 1.n1n2n3.

Other objects and advantages will become apparent to those skilled in the art upon review of the descriptions and drawings that follow.

In accordance with a primary aspect of the present invention there is provided a wound care device or barrier having a substantially rigid frame with a broad base. The apparatus must be strong enough to support the weight of sheets, blankets and other items used to cover the patient. The base has at least two points of contact with the bed, table or chair that the patient is lying on or sitting in. These points of contact are connected to each other by the bridge-like frame and are dispersed widely enough so that the barrier is able to straddle the relevant body parts without touching them and remain upright under modest movements of the body and bedclothes.

In accordance with a secondary aspect of the present invention, the device or barrier is large enough to provide ample ventilation to the relevant portion of the body that it straddles while at the same time preventing direct physical contact between the bedclothes and the relevant portion of the aforementioned body part. At the same time, it must be small enough so that the body is able to easily heat a pocket of air created within the barrier and bedclothes.

In accordance with a tertiary aspect of the present invention, the device creates and maintains a pocket of air between the bedclothes and the body part and can facilitate the healing of an open wound on that body part both by ventilating the wound and by preventing the adhesion of the bedclothes to the healing wound. Since the pocket of air created by the barrier is small enough to be easily heated by the body, the patient is able to stay warm and thereby the patient's propensity to contract or exacerbate an illness is minimized.

In accordance with another aspect of the invention there is contemplated a tapered footprint and varied cross section to accommodate the fact that human limbs are mostly of tapered cross section, i.e., the top of a thigh has a greater cross section than it does right above the knee. It is also contemplated that a diameter or cross section ratio A/B equal 1.n1n2n3 or 1.n4n5n6 can provide an ordering data point for the inventive device.

### BRIEF DESCRIPTION OF THE DRAWINGS

The present invention will be better understood upon reference to the following drawings taken in combination with a detailed description of the preferred embodiment following this brief description of the drawings in which:

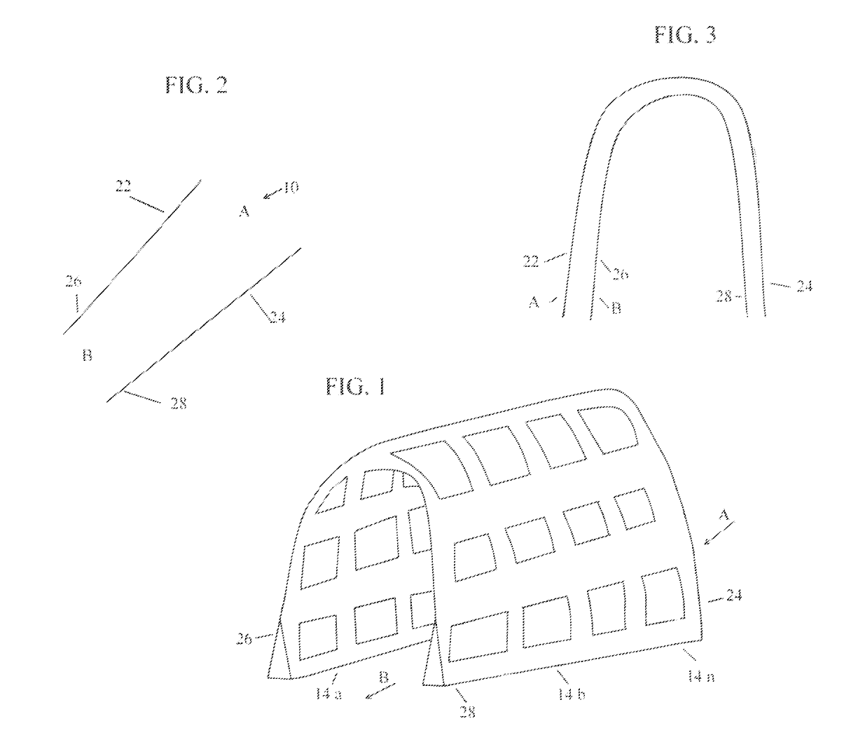

1. FIG. 1 is a rear perspective view of the preferred embodiment of the present invention.

2. FIG. 2 is a plan view of the footprint of the device of FIG. 1 showing generally that its lateral dimension at its opening end A is greater than at its exiting end B.

3. FIG. 3 is an exiting elevation view showing that the area of the entering end A is greater than of the exiting end B.

### DETAILED DESCRIPTION OF THE PREFERRED EMBODIMENT

As required, a detailed embodiment of the present invention is disclosed here in; however, it understood that the disclosed embodiment is merely exemplary of the invention which may be embodied in various forms. Therefore, specific structural and functional details disclosed herein are not to be interpreted as limiting, but merely as a basis for the claims and as a representative basis for teaching on skilled in the art to variously employ the present invention in virtually any appropriately detailed structure.

References now made to the drawings when like characteristics and features of the present invention are shown in the various figures being designated by the same reference numerals.

Wound care device 10 is comprised of a substantially rigid frame 12 and at least two points of contact 14a, 14b, . . . and 14n dispersed widely enough such that the barrier is able to straddle the relevant body parts without touching them and remain upright under modest movements of the body and bedclothes.

FIG. 2 is a footprint of the device 10 of FIG. 1. It shows an opening end A with an opening measured between points 22 and 24, and an exiting end B measured between point 26 and 28. The effect is to create a tapered diameter that can be measured A/B=(24−22)/(28−26).

FIG. 3 is elevation view in which the ratio A/B can be expressed as a ratio of diameters or a ratio of areas. These numeral relationships represent ordering data for a patient whose wound care can greatly benefit from the present invention.

While the invention has been described, disclosed, illustrated and shown in various terms or certain embodiments or modifications which it has assumed in practice, the scope of the invention is not intended to be, nor should it be deemed to be, limited thereby, and such other modifications or embodiments as may be suggested by the foregoing teachings herein are particularly reserved especially as they fall within the breath and scope of the claims here appended.

What is claimed is:

1. A wound care device comprising:
a substantially rigid frame having a cover disposed therein;
the frame having at least two points of contact with environmental features dispersed widely enough to permit the frame to straddle relevant body parts that are the site of a wound being treated.

2. The wound care device of claim 1 wherein the environmental features include portions of a patient's body surface in proximity to the wound being treated using the device.

3. The wound care device of claim 1 wherein the environmental features include aspects of a hospital bed in proximity to the wound being treated using the device.

4. The wound care device of claim 1 in which its diameter accommodates limb shape of a patient's body by tapering the diameter of the wound care device in accordance with a ratio that provides device ordering data, i.e., A/B equal 1.n1n2n3.

5. The wound care device of claim 1 in which its cross sectional area accommodates limb shape of a patient's body by tapering the cross sectional area of the wound care device in accordance with a ratio that provides device ordering data, i.e., A/B equal 1.n4n5n6.

6. The wound care device of claim 1 in which cover is one of a mesh, net, web, and lattice.
